(19) United States
(12) Patent Application Publication
(10) Pub. No.: US 2006/0222594 A1
(43) Pub. Date: Oct. 5, 2006

(54) Smart magnetic nanosphere preparation and manufacturing method thereof

(75) Inventors: Seung-Jun Lee, Daejeon (KR); Bong-Sik Jeon, Daejeon (KR); Jong-Duk Kim, Daejeon (KR); Yong-Min Hur, Seoul (KR); Ho-Taek Song, Incheon (KR); Jin-Suck Suh, Seoul (KR)

Correspondence Address: FISH & RICHARDSON PC, P.O. BOX 1022, MINNEAPOLIS, MN, 55440-1022, US

(73) Assignee: KOREA ADVANCED INSTITUTE OF SCIENCE AND TECHNOLOGY

(21) Appl. No.: 11/334,957

(22) Filed: Jan. 19, 2006

(30) Foreign Application Priority Data
Jan. 20, 2005 (KR) 10-2005-0005491

(51) Int. Cl. A61K 49/10; C07K 16/46

(52) U.S. Cl. 424/9.34; 977/930; 530/391.1

(57) ABSTRACT

Disclosed are a targeting magnetic nanosphere preparation capable of diagnosing and treating tumors in mammals and a method of manufacturing the same. The smart magnetic nanosphere preparation contains magnetic nano-sized iron oxide nanoparticles, which can be detected by magnetic resonance imaging (MRI), as a core material. The core material is encapsulated in a size of tens to hundreds of nanometers allowing particles to penetrate into deep areas of biological tissues using a biodegradable polymer having high affinity for biological tissues. The formed capsules are surface-modified with an antibody specific to a specific tumor. The smart magnetic nanosphere preparation can effectively diagnose tumors, as well as treat tumors because it has an antibody to a specific tumor.

### BACKGROUND OF THE INVENTION

1. Field of the Invention

The present invention relates to a smart magnetic nanosphere preparation and a method of manufacturing the same, and more particularly, to a targeting magnetic nanosphere preparation capable of diagnosing and treating tumors in mammals and a method of manufacturing the same.

The smart magnetic nanosphere preparation of the present invention contains magnetic nano-sized iron oxide nanoparticles, which can be detected by magnetic resonance imaging (MRI), as a core material. The core material is encapsulated in a size of tens to hundreds of nanometers allowing particles to penetrate into deep areas of biological tissues using a biodegradable polymer having high affinity for biological tissues. The resulting capsules are surface-modified with an antibody specific to a specific tumor. The targeting magnetic nanosphere preparation thus manufactured is effective in diagnosing and treating tumors.

2. Description of the Prior Art

Cancer, which is a malignant tumor, is the leading cause of death in the world, including Korea, along with heart-related diseases.

Cancer, which is the uncontrolled growth of malignant cells, occurs when abnormal cells derived from normal tissues or tumor cells, such as neoplastic cells that infinitely proliferate and form tumor masses, invade nearby tissues and eventually spread to local lymph nodes and distant organs via the blood or lymphatic system by a process called metastasis.

Unlike normal cells, cancer cells proliferate even under conditions in which normal cells do not grow. Cancer cells themselves emerge in a wide variety of forms, which are characterized by having several different degrees of invasive and offensive properties (Boring et al., CA Cancer J. Clin., 43:7 (1993)).

Many efforts have been made to find cell targets effective in diagnosing and treating tumors, and some research has intended to identify one or more transmembrane polypeptides or membrane-bound polypeptides, which are specifically expressed on the surface of one or more specific types of tumor cells relative to normal non-tumor cells.

Typically, membrane-bound polypeptides are more abundantly expressed on the surface of tumor cells than on the surface of non-tumor cells. Such tumor-associated cell surface antigen polypeptides can be identified and used for specifically targeting and destroying cancer cells through antigen immunotherapy.

Antigen immunotherapy has been proven to be very effective in treating several tumors. For example, Herceptin (registered trademark, Roche Korea Ltd.) is an antibody used to treat breast cancer. Also, Herceptin is a monoclonal antibody that selectively binds to the extracellular domain of human epidermal growth factor receptor 2 called HER2/neu, which is a proto-oncogene.

Overexpression of HER2/neu protein is found in about 25%-30% of primary breast cancer cases (Artemov D. et Al., Mag. Reson. Med., 49:409 (2003)).

A microcapsule or nanocapsule generally means a fine container that contains an active substance. Capsules protect a certain substance from surrounding environments, and function to allow their internal substances to display activity under conditions in which the internal substances are actually utilized, or to change the ability of their contents to pass through the gastrointestinal tract. Also, encapsulation is a technique that entraps solid-, liquid- or gas-phase active materials in a certain material or a system to control the release rate of their contents under specific conditions.

Typically, microcapsules or nanocapsules are prepared by interfacial polymerization, in situ polymerization, spray-drying or chilling, multiple emulsification, emulsification-diffusion, and the like.

In the present medical industry, to impart a drug with the ability to target an in vivo target site is called targeting. That is, “targeting” means any attempt to selectively direct drugs to act at sites where the drugs express their pharmaceutical effects. This is the most basic and important concept in the development of drug delivery system (DDS) strategies to achieve the optimization of drug administration through control of in vivo behavior of drugs.

As described above, “targeting” means any attempt to selectively direct drugs to act at sites where the drugs express their pharmaceutical effects. Such drug targeting can be achieved through surface modification of a specific molecule on the surface of nanospheres loaded with a drug. In detail, surface modification of nanospheres has been performed with the following aims: to selectively deliver a nanosphere preparation containing an effective ingredient to an in vivo specific site, to prevent a drug from being transported to an undesired site, to allow a drug to bypass passage barriers encountered during travel to a target site, to control drug delivery patterns, and to improve overall reproducibility and drug delivery efficiency.

### SUMMARY OF THE INVENTION

It is therefore an object of the present invention to provide a targeting smart magnetic nanosphere preparation and a manufacturing method thereof, the targeting smart magnetic nanosphere preparation being prepared by encapsulating magnetic iron oxide nanoparticles, which are detectable by magnetic resonance imaging (MRI), in a size of tens to hundreds of nanometers using a biodegradable polymer having high affinity for biological tissues, and surface-modifying the resulting capsules with an antibody specific to a specific tumor.

It is another object of the present invention to provide a method of diagnosing tumors, which is capable of diagnosing tumors using the targeting smart magnetic nanosphere preparation in which capsules are surface-modified with an antibody specific to a specific tumor.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic presentation of a smart magnetic nanosphere preparation according to an embodiment of the present invention, wherein (a) shows iron oxide nanoparticles, (b) shows a magnetic nanosphere containing iron oxide nanoparticles as a core material and having an outer shell consisting of PLGA, which encapsulates the core material, (c) is a TEM (transmission electron microscope) image of the magnetic nanosphere of (b), and (d) is a targeting smart magnetic nanosphere preparation in which a magnetic nanosphere is surface-modified with Herceptin;

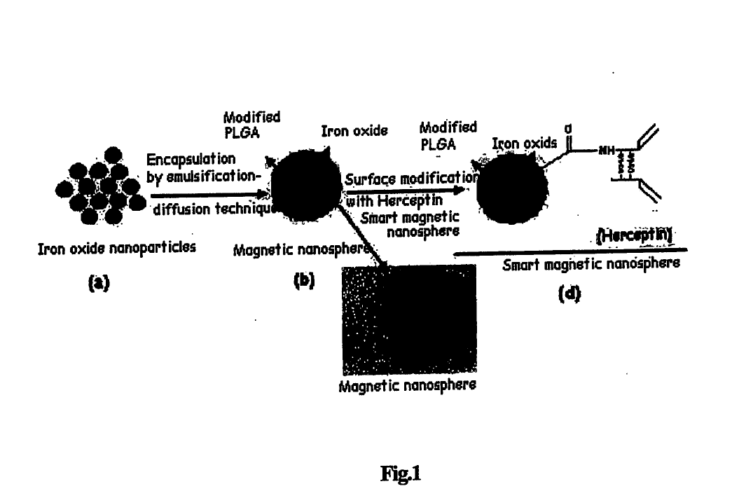

FIG. 2 is a graph showing the results of a tumor diagnostic test using a magnetic nanosphere preparation prepared in Example 1;

FIG. 3 is a graph showing the results of a tumor diagnostic test using a magnetic nanosphere preparation prepared in Example 2; and

FIG. 4 is a graph showing the potential of a magnetic nanosphere preparation prepared in Example 2 to serve as a probe for tumor diagnosis.

### DETAILED DESCRIPTION OF THE INVENTION

In order to accomplish the above objects, the present invention is directed to a smart magnetic nanosphere preparation in which capsules, composed of iron oxide as a core material and a biodegradable polymer encapsulating the iron oxide, are bound to an antibody on their surface.

In the present invention, due to its physiochemical property of responding to the intensity of the MRI's magnetic field, iron oxide serves as an effective probe, which can image applied tissues by MR imaging when administered to deep areas of biological tissues. In the present invention, such iron oxide may be either one selected from among Fe₃O₄ and γ-Fe₂O₃, which have a particle size of 1-10 nm.

When iron oxide having a particle size less than 1 nm is used, clustering of particles occurs. When iron oxide having a particle size exceeding 10 nm is used, nanoencapsulation does not progress well. Thus, iron oxide used in the preparation of the magnetic nanosphere preparation of the present invention preferably has a particle size of 1-10 nm.

The biodegradable polymer used in the present invention may be any one that is not harmful to the body, has high affinity to human tissues and is able to penetrate into the deep areas of human tissues.

The biodegradable polymer useful in the present invention may be any one selected from among polylactide (PLA), polyglycolide (PGA) and their copolymer, poly(D,L lactide-co-glycolide) (PLGA). PLGA may be prepared by copolymerizing PLA and PGA at a ratio of 5:5-7:3 and may have a molecular weight of 50,000-150,000.

The biodegradable polymer, used as a shell material of capsules, may be modified by esterification of a terminal group thereof in order to bind an antibody to the surface of capsules. In a preferred embodiment for the modification of a biodegradable polymer, when PLGA is used as the biodegradable polymer, a terminal group of PLGA may be modified in a succinimide ester form through reactions with N-hydroxysuccinimide (NHS) and 1,3-dicyclohexylcarbodiimide (DCC).

An antibody capable of diagnosing and treating tumors may be bound to the surface of capsules, which is composed of a biodegradable polymer, to generate a desired smart magnetic nanosphere preparation.

The antibody used in the present invention may be an antibody against a tumor to be diagnosed and/or treated.

The antibody useful in the present invention may be either one selected from among Herceptin and Trastuzumab, which are widely used as antibodies against breast cancer.

In another aspect, the present invention includes a method of manufacturing a smart magnetic nanosphere preparation in which an antibody is bound to the surface of capsules composed of iron oxide as a core material and a biodegradable polymer encapsulating the iron oxide.

The present method of manufacturing a smart magnetic nanosphere preparation provides capsules to which an antibody is bound, the method comprising the steps of:

(1) dissolving a biodegradable polymer in a solvent, esterifying a terminal group of the biodegradable polymer, adding iron oxide to the solution and dispersing the iron oxide;

(2) saturating the iron oxide-dispersed solution with an aqueous solution and emulsifying the saturated solution by agitation;

(3) adding water to the emulsified solution to diffuse the solvent into the aqueous phase in order to generate capsules containing the iron oxide as a core material and the biodegradable polymer encapsulating the iron oxide; and

(4) binding an antibody to the surface of the capsules.

Hereinafter, the present method of manufacturing a magnetic nanosphere preparation will be described in more detail with respect to each step.

(1) The step of dissolving a biodegradable polymer in a solvent, esterifying a terminal group of the biodegradable polymer and adding iron oxide to the solution to be dispersed

Examples of the biodegradable polymer useful in the present invention include polylactide (PLA), polyglycolide (PGA), and their copolymer, poly(D,L lactide-co-glycolide) (PLGA). In order to bind an antibody to the surface of capsules formed, the biodegradable polymer is preferably modified by esterification at a terminal group thereof before being mixed with iron oxide after being dissolved in a solvent.

In a preferred embodiment for the modification of a biodegradable polymer, when PLGA is used as the biodegradable polymer, PLGA is dissolved in a solvent and allowed to react with N-hydroxysuccinimide (NHS) and 1,3-dicyclohexylcarbodiimide (DCC), thereby modifying a terminal group of PLGA with succinimide ester. Herein, PLGA is dissolved in a solvent at a PLGA:solvent weight ratio of 1:5-10. A mixture of NHS and DCC is added at a molar ratio of 1.0-3.0 to PLGA and is allowed to react with PLGA, thereby generating a biodegradable polymer, PLGA, which is modified with succinimide ester at its terminal group.

PLGA serves as a solute for a solvent. When a solute is used at a very small weight ratio or in an excessive amount, it negatively affects chemical reactions. Thus, PLGA is preferably dissolved in a solvent at a PLGA to solvent weight ratio of 1:5-10.

NHS and DCC used when a terminal group of PLGA is modified with succinimide ester may be mixed at an NHS to DCC ratio of 1-9:9-1. When the NHS/DCC mixture is used in a small or excessive amount, reactions do not progress well, or NHS and DCC remain after reactions. Thus, the NHS/DCC mixture is preferably added at a molar ratio of 1.0-3.0 to PLGA.

A solvent available for the dissolution of a biodegradable polymer may be any one that is not harmful to the body, is chemically and physically stable, is partially water-miscible and in which high molecular weight molecules are highly soluble. The solvent useful in the present invention may be any one selected from among propylene carbonate (PC), ethyl acetate (EA), methylene chloride (MC) and dimethyl sulfoxide (DMSO). For example, 0.25±0.01 ml of PC, 0.1±0.01 ml of EA and 0.5±0.01 ml of MC can be dissolved in 1 g of water.

Iron oxide is added to and dispersed in the solution in which a biodegradable polymer modified with succinimide ester at a terminal group thereof is dissolved. Iron oxide serves as an effective probe, which can image applied tissues when used with MR imaging. In the present invention, such iron oxide may be either one selected from among Fe₃O₄ and γ-Fe₂O₃, which have a particle size of 1-10 nm, and may be used in an amount of 0.1-5.0 wt % based on the weight of the biodegradable polymer. When iron oxide is used in an amount less than 0.1 wt % based on the weight of the biodegradable polymer, it does not serve as a probe because formed capsules have a low iron oxide content. When iron oxide is used in an amount exceeding 5.0 wt %, iron oxide particles aggregate, leading to an increase in capsule size. Thus, in the present invention, iron oxide is preferably added to the solution, in which a biodegradable polymer modified with succinimide ester at a terminal group thereof is dissolved, in an amount of 0.1-5.0 wt % based on the weight of the biodegradable polymer.

After iron oxide is added to the biodegradable polymer-containing solution, the solution may be sonicated at 20-50 kHz and 50-100 W for 5-15 min in order to disperse iron oxide. When the sonication was carried out under various conditions in order to increase the dispersion of iron oxide, it was found that the sonication is preferably carried out under the aforementioned conditions.

(2) The step of saturating the iron oxide-dispersed solution with an aqueous solution in which a stabilizing agent is dissolved and emulsifying the saturated solution by agitation

The iron oxide-dispersed solution obtained at Step (1) is saturated with an aqueous solution in which a stabilizing agent is dissolved, and is emulsified.

The stabilizing agent may be one or more selected from among Tween 20 (polyoxyethylene sorbitan monolaurate), NP-9, pluronics, sodium lauryl sulfate (SDS), Dowfax 2A1 (sodium dodecyl diphenyloxide disulfonate) and polyvinyl alcohol (PVA).

The stabilizing agent may be added to water in an amount of 0.5-30 wt % based on the weight of water. When the stabilizing agent is used in an amount less than 0.5 wt % or exceeding 30 wt % based on the weight of water, it does not function as a parameter for stability, and surplus molecules act as impurities and negatively affect the formation of fine particles.

After the iron oxide-dispersed solution is saturated with an aqueous solution in which a stabilizing agent is dissolved, the saturated solution is emulsified using a homogenizer. Herein, the homogenizer is used to force the saturated aqueous solution to agitate and emulsify because the solvent used to dissolve a biodegradable polymer is not spontaneously emulsified in water. The emulsification using a homogenizer is preferably carried out at an agitation rate of 5000-20000 rpm for 5-15 min to agitate the saturated solution. As the homogenizer's rotation speed increases, greater shearing force is applied to emulsion droplets for a predetermined time. In this case, the droplets have a decreased size, and thus, fine particles generated from the droplets have a decreased size. When the agitation rate exceeds 20000 rpm, aggregation of fine particles occurs.

(3) The step of adding water to the emulsified solution to diffuse the solvent into the aqueous phase and generate capsules containing the iron oxide as a core material and the biodegradable polymer encapsulating the iron oxide Water was added to the emulsified solution obtained at Step (2) to diffuse the organic solvent in the emulsified solution into the aqueous phase, thereby forming magnetic nanosphere capsules which have a spherical shape 60-200 nm in size and contain iron oxide as a core material, encapsulated by the biodegradable polymer.

The water added to the emulsified solution is determined according to the solubility of the organic solvent in the emulsified solution in water. Water at 10-60° C. may be used.

(4) The step of binding an antibody to the surface of the capsules

The magnetic nanosphere capsules, obtained at Step (3), which contain iron oxide as a core material, encapsulated by the biodegradable polymer, are allowed to react with an antibody to couple the antibody to the biodegradable polymer constituting the outer shell of the capsules, thereby generating a magnetic nanosphere preparation in which an antibody is coupled to the capsule surface.

The antibody reacted with the biodegradable polymer comprising the outer shell of capsules may be an antibody against a tumor to be diagnosed and/or treated. Examples of the antibody useful in the preparation of the magnetic nanosphere preparation of the present invention include Herceptin and Trastuzumab, which are widely used as antibodies against breast cancer.

The magnetic nanosphere capsules are reacted with an antibody as follows. First, the magnetic nanosphere capsules are dispersed in a buffer solution of pH 7-8. The antibody is added to the buffer solution at a capsule:antibody molar ratio of 1:0.5-5, and is allowed to react at 4-10° C. for 12-24 hrs, thereby generating a magnetic nanosphere preparation in which the antibody is coupled to the surface of magnetic nanospheres and which has a size of 65-250 nm. When the magnetic nanosphere preparation was prepared under various conditions, it was found that the magnetic nanosphere preparation is preferably prepared under the aforementioned conditions. For example, with respect to reaction temperature, the reaction between the magnetic nanospheres and an antibody may be carried out at 4-10° C. to prevent a decrease in antibody activity due to the instability of the antibody at high temperature.

In a further aspect, the present invention includes a method of diagnosing tumors using the magnetic nanosphere preparation. When the magnetic nanosphere preparation conjugated to an antibody is injected into the body and the body is subjected to MR imaging, MR images for a tumor of interest can be obtained due to iron oxide contained in the nanosphere preparation if a subject has a tumor antigen to which the antibody is able to bind. In this way, tumor incidence can be diagnosed.

A better understanding of the present invention may be obtained through the following examples and test examples which are set forth to illustrate, but are not to be construed as the limit of the present invention.

### EXAMPLE 1

(1) 4 g of poly(D,L lactide-co-glycolide) (PLGA), a 1:1 copolymer of polylactide (PLA) polyglycolide (PGA), were dissolved in 30 ml of dimethylsulfoxide (DMSO) at room temperature, and mixed with a 1:1 mixture of N-hydroxysuccinimide (NHS) and 1,3-dicyclohexylcarbodiimide (DCC) at a molar ratio of 1.5 to PLGA, followed by reaction for 18 hrs. The polymer was precipitated using ethanol (Sigma Aldrich) to recover PLGA modified with succinimide ester at its terminal group.

(2) 70 mg of PLGA modified with succinimide ester at its terminal group at Step (1) were dissolved in 3.5 ml of ethyl acetate (EA). 23 mg of iron oxide nanoparticles having a size of 3-5 nm were added to the PLGA-containing solution, and the resulting solution was sonicated (25 kHz, 80 W) to disperse the iron oxide.

(3) In order to prepare an emulsion, 330 mg of pluronic F-127 were dissolved in 10 ml of water at room temperature, and mixed with the iron oxide nanoparticle-dispersed solution obtained at Step (2). The mixture was then emulsified at 15000 rpm for 7 min using a homogenizer.

(4) 40 ml of water at 20° C. was added to the emulsion obtained at Step (3) to diffuse the organic solvent into water, thereby forming magnetic nanospheres in a capsule form containing iron oxide nanoparticles as a core material and PLGA as an outer shell encapsulating the iron oxide.

(5) 15 mg of the magnetic nanospheres obtained in Steps (1) to (4) were dispersed in 3 ml of a buffer (PBS, pH 7.4), mixed with 8 mg of Herceptin, and allowed to react with Herceptin at 10° C. for 24 hrs, thereby generating a magnetic nanosphere preparation in which magnetic nanospheres were bound to Herceptin on the surface thereof. The binding of Herceptin to the surface of magnetic nanospheres was achieved by a chemical reaction between the magnetic nanosphere surface coupled with succinimide ester and an amine group (NH₂) of the Herceptin antibody.

### EXAMPLE 2

(1) 8 g of PLGA, a 1:1 copolymer of PLA and PGA, were dissolved in 60 ml of DMSO at room temperature, and mixed with a 1:1 mixture of NHS and DCC at a molar ratio of 1.5 to PLGA, followed by reaction for 18 hrs. The polymer was precipitated using ethanol (Sigma Aldrich) to recover PLGA modified with succinimide ester at its terminal group.

(2) 200 mg of PLGA modified with succinimide ester at its terminal group at Step (1) were dissolved in 10 ml of EA. 60 mg of 3-5-nm iron oxide nanoparticles were dispersed in the PLGA solution by sonication at 25 kHz and 80 W.

(3) In order to prepare an emulsion, 1 g of pluronic F-68 were dissolved in 20 ml of water at room temperature, and mixed with the iron oxide nanoparticle-dispersed solution obtained at Step (2). The mixture was then emulsified at 15000 rpm for 8 min using a homogenizer.

(4) 90 ml of water at 30° C. were added to the emulsion obtained at Step (3) to diffuse the organic solvent into water, thereby forming magnetic nanospheres in a capsule form containing iron oxide nanoparticles as a core material and PLGA forming an outer shell by encapsulating the iron oxide.

(5) 15 mg of the magnetic nanospheres obtained from Steps (1) to (4) were dispersed in 3 ml of a buffer (PBS, pH 7.4), mixed with 15 mg of Herceptin, and allowed to react with Herceptin at 7° C. for 24 hrs, thereby generating a magnetic nanosphere preparation in which magnetic nanospheres were bound to Herceptin on the surface thereof. The binding of Herceptin to the surface of magnetic nanospheres was achieved by a chemical reaction between the magnetic nanosphere surface coupled with succinimide ester and an amine group (NH₂) of the Herceptin antibody.

### TEST EXAMPLE 1

A tumor diagnostic test was performed using the magnetic nanosphere preparation, prepared in Example 1, in which the surface of magnetic nanospheres is conjugated with Herceptin antibody.

In order to assess the targeting ability of the magnetic nanospheres, the magnetic nanosphere preparation in which the surface of magnetic nanospheres is conjugated with Herceptin antibody was added to HER2/neu-overexpressing NIH3T6.7 cells (also referred to simply as T6.7 cells herein, Diagnostic Radiology Lab, College of Medicine, Yonsei University, Korea) and HER2/neu-nonexpressing NIH3T3 cells (also referred to simply as T3 cells herein, Diagnostic Radiology Lab, College of Medicine, Yonsei University).

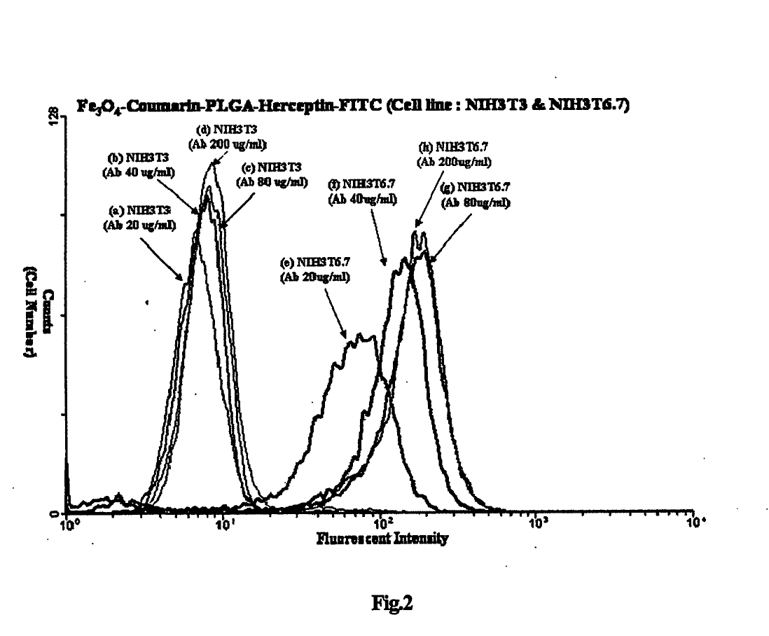

The magnetic nanosphere preparation with which the Herceptin antibody was conjugated at 20 μg/ml, 40 μg/ml, 80 μg/ml and 200 μg/ml was added to NIH3T6.7 cells and NIH3T3 cells. After the cells were incubated for 30 min to 60 min, they were washed three to five times. During the washing, the magnetic nanosphere preparation was washed off of HER2/neu-nonexpressing NIH3T3 cells, whereas it remained on HER2/neu-overexpressing NIH3T6.7 cells due to the antigen-antibody reaction between Herceptin and HER2/neu. As described above, an in vitro targeting test was performed, and the results are given in FIG. 2.

The targeting property of the magnetic nanosphere preparation was measured using a fluorescence activated cell sorter (FACS, Clinical Medical Center, College of Medicine, Yonsei University).

As shown in FIG. 2, FACS data for the in vitro-targeted HER2/neu-overexpressing T6.7 cells and HER2/neu-nonexpressing T3 cells revealed that the peak for T6.7 cells was shifted to the right compared to that for T3 cells. Also, when the concentration of Herceptin increased, the peak in FACS data was further shifted to the right due to the increased targeting of nanospheres to T6.7 cells. These results indicate that the magnetic nanospheres are targeted to target cells by binding to HER2/neu expressed on the surface of T6.7 cells through the Herceptin antibody conjugated with their surface.

### TEST EXAMPLE 2

A tumor diagnostic test was performed using the magnetic nanosphere preparation, prepared in Example 2.

In order to assess the targeting property of the magnetic nanospheres, the magnetic nanosphere preparation in which the surface of magnetic nanospheres is conjugated with Herceptin antibody was added to HER2/neu-overexpressing NIH3T6.7 cells (also referred to simply as T6.7 cells herein, Diagnostic Radiology Lab, College of Medicine, Yonsei University) and HER2/neu-nonexpressing BxPC-3 cells (also referred to simply as PC-3 cells herein, Diagnostic Radiation Lab, College of Medicine, Yonsei University).

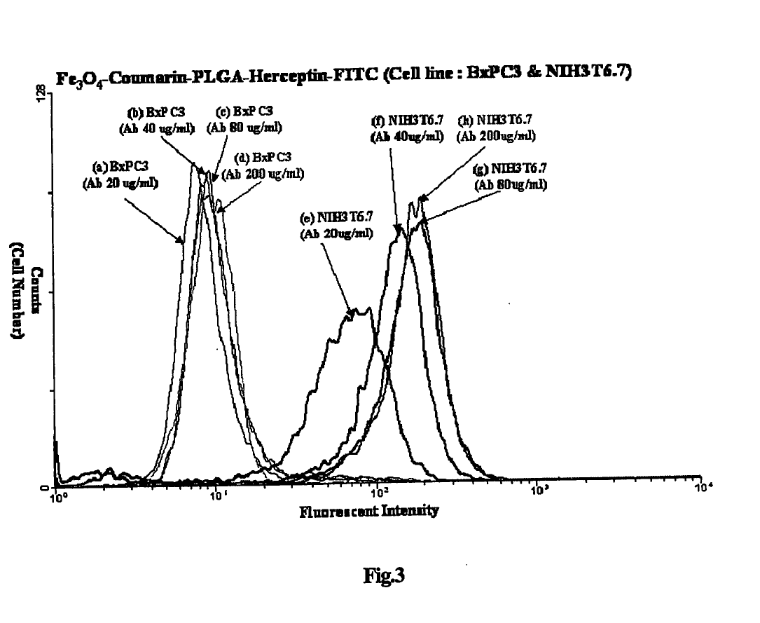

The magnetic nanosphere preparation with which the Herceptin antibody was conjugated at 20 μg/ml, 40 μg/ml, 80 μg/ml and 200 μg/ml was added to NIH3T6.7 cells and BxPC-3 cells. After the cells were incubated for 30 min to 60 min, they were washed three to five times. During the washing, the magnetic nanosphere preparation was washed off of HER2/neu-nonexpressing PC-3 cells, whereas it remained on HER2/neu-overexpressing NIH3T6.7 cells due to the antigen-antibody reaction between Herceptin and HER2/neu. As described above, an in vitro targeting test was performed, and the results are given in FIG. 3.

The targeting property of the magnetic nanosphere preparation was measured using a fluorescence activated cell sorter (FACS, Clinical Medical Center, College of Medicine, Yonsei University).

As shown in FIG. 3, FACS data for the in vitro-targeted HER2/neu-overexpressing T6.7 cells and HER2/neu-nonexpressing PC-3 cells revealed that the peak for T6.7 cells was shifted to the right compared to that for PC-3 cells. Also, when the concentration of Herceptin increased, the peak in FACS data was further shifted to the right due to the increased targeting of nanospheres to T6.7 cells. These results indicate that the magnetic nanospheres are targeted to target cells by binding to HER2/neu expressed on the surface of T6.7 cells through the Herceptin antibody conjugated with their surface.

### TEST EXAMPLE 3

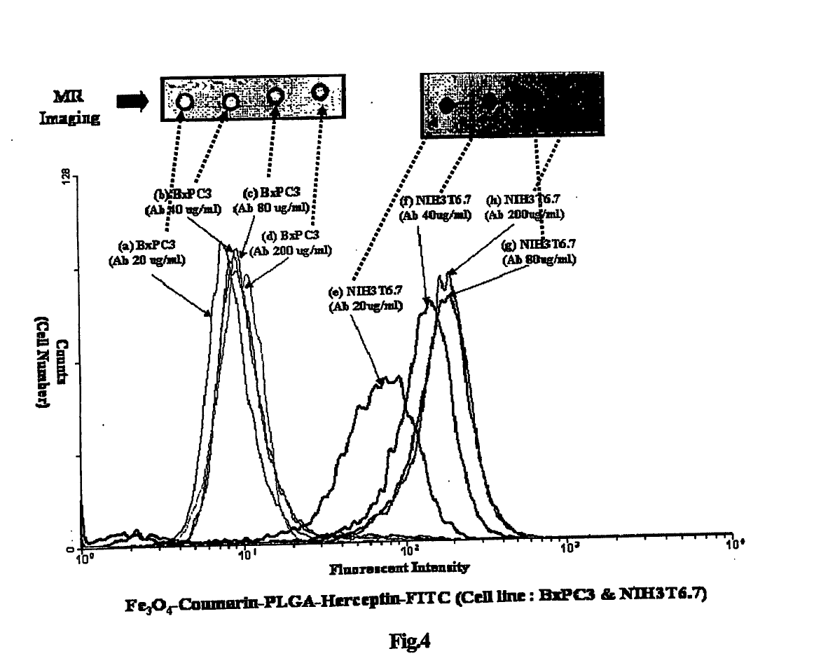

In order to determine whether the magnetic nanosphere preparation prepared in Example 2 can be used as a probe for tumor diagnosis, an in vitro test was performed with MRI (1.5 T, GE, Dept. of Diagnostic Radiology, Severance Hospital, College of Medicine, Yonsei University). The results are given in FIG. 4.

Cells were treated with the magnetic nanosphere preparation, harvested and tracked in vitro with MR imaging. As shown in FIG. 4, when MR imaging was performed for (e), (f), (g) and (h) samples from HER2/neu-overexpressing T6.7 cells and (a), (b), (c) and (d) samples from HER2/neu-nonexpressing PC-3 cells, MR images of T6.7 cells were found to be black compared to those of PC-3 cells.

In addition, when the concentration of Herceptin increased, the MR images of T6.7 cells were darkened due to the increased targeting of magnetic nanospheres to T6.7 cells. The black images were generated when the Herceptin antibody bound to the surface of the magnetic nanosphere preparation was targeted to T6.7 cells by forming complexes with HER2/neu expressed on T6.7 cells of (e), (f), (g) and (h) samples, and iron oxide, remaining on the cell surface through the antigen-antibody complexes, responded to MR imaging. In contrast, since PC-3 cells of (a), (b), (c) and (d) samples did not express HER2/neu and thus did not bind to the Herceptin antibody conjugated to the surface of the magnetic nanosphere preparation, they did not display black MR images.

These results indicate that the magnetic nanosphere preparation is targeted to target cells by binding to HER2/neu expressed on the surface of T6.7 cells through the Herceptin antibody conjugated with their surface. Therefore, the magnetic nanosphere preparation of the present invention is useful for tumor diagnosis.

As apparent from the results of Test Examples, the smart magnetic nanosphere preparation of the present invention is able to effectively diagnose tumors.

In addition, the magnetic nanosphere preparation of the present invention is able to treat tumors as well as diagnose tumors because it has an antibody to a specific tumor.

While the invention has been described in detail and with reference to specific examples thereof, it will be apparent to one skilled in the art that various changes and modifications can be made therein without departing from the spirit and scope of the invention as disclosed in the accompanying claims.

1. A smart magnetic nanosphere preparation in which capsules are composed of iron oxide as a core material and a biodegradable polymer encapsulating the iron oxide and are bound to an antibody on a surface thereof.

2. The smart magnetic nanosphere preparation as set forth in claim 1, wherein the iron oxide is either one selected from among Fe₂O₄ and γ-Fe₂O₃.

3. The smart magnetic nanosphere preparation as set forth in claim 1, wherein the biodegradable polymer is any one selected from among polylactide, polyglycolide and poly(lactide-co-glycolide).

4. The smart magnetic nanosphere preparation as set forth in claim 1, wherein the antibody is either one selected from among Herceptin and Trastuzumab.

5. A method of manufacturing a smart magnetic nanosphere preparation, comprising the steps of:
(1) dissolving a biodegradable polymer in a solvent, esterifying a terminal group of the biodegradable polymer, adding iron oxide to the resulting solution and dispersing the iron oxide;
(2) saturating the iron oxide-dispersed solution with an aqueous solution and emulsifying the saturated solution by agitation;
(3) adding water to the emulsified solution to diffuse the solvent into the aqueous phase in order to generate capsules containing the iron oxide as a core material and the biodegradable polymer encapsulating the iron oxide; and
(4) binding an antibody to a surface of the capsules.

6. The method of manufacturing the smart magnetic nanosphere preparation as set forth in claim 5, wherein the biodegradable polymer is any one selected from among polylactide, polyglycolide and poly(lactide-co-glycolide).

7. The method of manufacturing the smart magnetic nanosphere preparation as set forth in claim 5, wherein the solvent used to dissolve the biodegradable polymer is any one selected from among propylene carbonate, ethyl acetate, methylene chloride and dimethyl sulfoxide.

8. The method of manufacturing the smart magnetic nanosphere preparation as set forth in claim 5, wherein the iron oxide is either one selected from among Fe₃O₄ and γ-Fe₂O₃.

9. The method of manufacturing the smart magnetic nanosphere preparation as set forth in claim 5, wherein the aqueous solution includes a stabilizing agent selected from among Tween 20, NP-9, pluronics, sodium lauryl sulfate, Dowfax 2A1 and polyvinyl alcohol.

10. The method of manufacturing the smart magnetic nanosphere preparation as set forth in claims 5, wherein the capsules are 60-200 nm in diameter.

11. The method of manufacturing the smart magnetic nanosphere preparation as set forth in claim 5, wherein the antibody is either one selected from among Herceptin and Trastuzumab.

12. A method of diagnosing tumors using the smart magnetic nanosphere preparation of claim 1.

13. A method of diagnosing tumors using the smart magnetic nanosphere preparation of claim 2.

14. A method of diagnosing tumors using the smart magnetic nanosphere preparation of claim 3.

15. A method of diagnosing tumors using the smart magnetic nanosphere preparation of claim 4.
